(19) United States
(12) Patent Application Publication
(10) Pub. No.: US 2014/0031668 A1
(43) Pub. Date: Jan. 30, 2014

(54) Surgical and Medical Instrument Tracking Using a Depth-Sensing Device

(71) Applicants: Jean-Pierre Mobasser, Indianapolis, IN (US); Eric Potts, Indianapolis, IN (US); Dean Karahalios, Lake Forest, IL (US)

(72) Inventors: Jean-Pierre Mobasser, Indianapolis, IN (US); Eric Potts, Indianapolis, IN (US); Dean Karahalios, Lake Forest, IL (US)

(73) Assignee: DISRUPTIVE NAVIGATIONAL TECHNOLOGIES, LLC, Longmont, CO (US)

(21) Appl. No.: 13/821,699

(22) PCT Filed: Sep. 6, 2011
(86) PCT No.: PCT/US11/50509

§ 371 Date: Aug. 19, 2013

Related U.S. Application Data
(60) Provisional application No. 61/380,923, filed on Sep. 8, 2010.

(51) Int. Cl. A61B 19/00; A61B 5/055; A61B 7/00; A61B 5/00; A61B 6/12; A61B 5/06

(52) U.S. Cl. CPC A61B 19/5244; A61B 6/12; A61B 5/062; A61B 7/00; A61B 5/0075; A61B 5/055
USPC 600/409; 600/424; 600/417

(57) ABSTRACT

A motion-sensing mechanism is provided that facilitates numerous aspects of the medical industry. In one aspect, the motion-sensing mechanism is used to track instruments and personnel in a field of view relative to a patient such that the instrument or personnel may be displayed on a heads-up display showing a model of the patient's anatomy. In another aspect, the motion-sensing mechanism makes a reference image of a patient, visitor, or staff such that when the subject of the reference images passes another (or the same) motion-sensing mechanism, the identity of the subject is determined or recognized. In still other aspects, the motion-sensing mechanism monitors motion of a portion of a patient's anatomy and compares the same to an expected motion for diagnostic evaluation of pain generators, physical therapy effectiveness, and the like.

### CLAIM OF PRIORITY UNDER 35 U.S.C. §119

This application claims priority under 35 U.S.C. §119(e) to U.S. Provisional Patent Application No. 61/380,823, filed Sep. 8, 2010, titled Surgical and Medical Instrument Tracking Using a Depth-Sensing Device.

### CLAIM OF PRIORITY UNDER 35 U.S.C. §120

None.

### REFERENCE TO CO-PENDING APPLICATIONS FOR PATENT

None.

### BACKGROUND

1. Field

The technology of the present application relates generally to medical devices and methods and, more specifically, to tracking medical and surgical instruments, personnel, patients, surgical navigation, and/or anatomical features, positions, and movements of a patient in three dimensions using image guided navigation equipped with motion-sensing mechanism depth-sensing devices.

2. Background

The accurate and precise identification of anatomical structures during surgery is critical in performing safe and effective operative procedures. Traditionally, surgeons have relied on direct visualization of the patient's anatomy to safely maneuver surgical instruments in and around critical structures. The accuracy and precision of these maneuvers may be suboptimal, leading to complications. In addition, a surgeon only can visualize what is on the surface of the anatomy that has been exposed. Structures not exposed and immediately visible are at risk to error. A surgeon relies on their perception of the patient's anatomy to avoid harm or damage to unseen, and in some cases seen, patient organs and the like. Even with considerable experience, there remains a significant risk of human error.

In view of the risks, computer assisted surgery or surgical navigation technology has developed. Using current technology, the most important component of computer assisted surgery is the development of the model of the patient's anatomy and the referencing of the anatomy for the introduction of an instrument. A number of medical imaging technologies can be used to create the computer model of the patient's anatomy. One exemplary technology includes, for example, computed tomography (“CT”—sometimes referred to as a CAT) scans can be used to image a patient's anatomy. CT uses a large number of 2-dimensional x-ray pictures to develop a 3-dimensional computer image of the x-rayed structure. Generally, the x-ray machine has a C-shaped arm that extends around the body of the patient to take x-ray slices of the patient. The x-ray source on one side with the x-ray sensors on the other. The x-ray slices or cross-sections of the patient are combined using a conventional tomographic reconstruction process to develop the image used for the surgical navigation. Another exemplary technology includes, for example, magnetic resonance imaging (“MRI”) to image the patient's anatomy. The MRIs may be stacked using a conventional algorithm to generate a 3-dimensional image of the patient's anatomy. These are but two examples of generating a 3-dimensinal image of a patient's anatomy.

One exemplary procedure occurs in cranial neurosurgical procedures where a surgeon has traditionally needed to have a very keen understanding of a patient's pathology relative to the complex three-dimensional anatomy of the brain. The brain pathology may be depicted in pre-operative imaging studies obtained using CT scans or MRIs. While the imaging provides details regarding the pathology, the images are not self orienting. Thus, procedures are complicated by the need to reference the image to the actual position of the patient (described more below). Moreover, additional complications arise because the position of the patient and the pathology may shift during the course of an operative procedure, again compromising the precision of the surgeon's perception of the pathology and location of the target.

Additional challenges are faced in spinal procedures where the inherent flexibility of the spine changes the position of targets planned for decompression or resection as seen on pre-operative imaging studies. This typically requires obtaining intra-operative radiographic imaging to localize targets. In addition, the need to implant instrumentation poses challenges to the surgeon. Insertion of devices into the spine using anatomical landmarks is associated with certain degrees of inaccuracy. These inaccuracies are compounded by the inability to visualize the necessary path or target of an implant through the spine. This is further compounded in minimally invasive procedures, where overlying skin and soft tissue further inhibit visual inspection. Again, conventional intraoperative imaging using plain radiographs or fluoroscopy improves accuracy and precision but has limitations.

Intraoperative image guided navigation allows the surgeon to accurately and precisely determine the position of surgical instruments relative to the patient's anatomy. The precise position of the tip of a surgical instrument is displayed on a computer monitor overlying the radiographic image of the patient's anatomy. The location of the instrument relative to anatomic structures may be depicted in multiple two-dimensional planes or in three-dimensions. This allows the surgeon to operate in and around critical structures with greater accuracy and precision. In addition, the position of instruments relative to deeper underlying structures that are not visible becomes possible. This allows the surgeon to avoid injuring organs and tissue as well as navigate instruments to deeper targets with smaller incisions as the surgeon does not need to see the organ or tissue.

In order to accomplish image-guided navigation, the instruments and the patient's anatomy must be recognized, the relative positions to each other registered, and the subsequent motion tracked and displayed on the overhead monitor. Navigation systems to date have relied on several methods for tracking. The methods include articulated arms with position sensors that are attached to the patient's anatomy, infrared cameras that track light emitting diodes (EDs) or reflective spheres attached to the instruments and to the patient's anatomy and systems that track the position of an antenna attached to the instruments within a magnetic field generated around the patient's anatomy.

Recognition of specific instruments requires that additional devices are fitted onto instruments, including unique arrays of LEDs or reflective spheres for infrared systems or antennas in the case of magnetic field technology. This limits the ability to use many instruments that a surgeon may want to use during any procedure. Furthermore, the fitting of these additional devices may significantly change the ergonomics of a surgical instrument, thus limiting its utility. Furthermore, the recognition of the attached devices requires that the specific dimension or quality of the device are pre-programmed into the computer processor, again limiting the ability to track only those instruments fitted with secondary devices that are “known” to the computer.

As mentioned above, one component necessary for the use of surgical navigation technologies is registration. Registration involves identifying structures in the pre-operative scan and matching them to the patient's current position in the operation setting as well as any changes in that position. Registration may include placing at known locations markers. Such markers may include, for example, bone screws, a dental splint, or reference markers attached to the skin. Other types of registration do not use markers, but rather surface recognition of the patient, such as using, for example, a laser surface scanning system to match points on the skin during the imaging to the points in the operating room.

Once the patient orientation relative to the images is established, registration further requires that the relative position of an instrument to be tracked is established relative to the patient's anatomy. This may be accomplished by a manual process whereby the tip of the instrument is placed over multiple points on the patient's anatomy, and the tip is correlated to the known location of the points on the patient's pre-operative imaging study. The registration process tends to be a cumbersome and time-consuming process, and is compromised by the inaccuracy or human error inherent in the surgeon's ability to correlate the anatomy. Automatic registration involves obtaining real-time intraoperative imaging with additional referencing devices attached to the patient's anatomy. Once the imaging is completed, the attached devices are referenced relative to the patient's anatomy. This is a marked improvement over manual registration, but requires additional intra-operative imaging which is time consuming, expensive, and exposes the patient and operating room personnel to additional radiation exposure.

Tracking solutions to date have a number of shortcomings. Radiogrpahic imaging techniques, such as fluoroscopy, involve the use of x-rays and carry with them certain health risks associated with exposure to ionizing radiation, both to patients and operating room personnel. Fluoroscopes also may be subject to image blurring with respect to moving objects due to system lag and other operating system issues. Articulated arms, moreover, are cumbersome and despite multiple degrees of freedom, these devices are constrained in their ability to reach certain anatomic points. As such, they pose ergonomic challenges in that they are difficult to maneuver. In addition, the tool interfaces are limited and cannot be applied to the use of all instruments a surgeon may desire to use. Infrared camera tracking provides significantly more flexibility in the choice and movement of instruments, but obstruction of the camera's view of the LEDs or reflective spheres leads to lapses in navigation while the line-of-sight is obscured. Magnetic field-based tracking overcomes the line-of-sight problem, but is susceptible to interference from metal instruments leading to inaccuracy.

All of the commonly used tracking systems mentioned can only track objects that are fitted with or attached to additional devices such as mechanical arms, LEDs, reflective spheres, antennas, and magnetic field generators. This precludes the ability to use some instruments available in a surgical procedure.

Thus, against this background, there is a need to provide improved navigational procedures that improve the ability to track instruments and the patient with respect to the image established pre-operatively.

### SUMMARY

This Summary is provided to introduce a selection of concepts in a simplified and incomplete manner highlighting some of the aspects further described in the Detailed Description. This Summary, and the foregoing Background, is not intended to identify key aspects or essential aspects of the claimed subject matter. Moreover, this Summary is not intended for use as an aid in determining the scope of the claimed subject matter.

In some aspects of the technology of the present application, provides a motion-sensing mechanism to track multiple objects in a field of view associated with a surgical site. The track objects are superimposed to a display of a model of the patient's anatomy to enhance computer assisted surgery or surgical navigation surgery.

In other aspects of the technology, the motion-sensing mechanism locates maps the patient's topography, such as, for example, the contour of the patient's skin. A processor receives images of the patient's pathology using computer tomography or magnetic resonance imaging and aligns to generate a model of the patient's pathology. The processor aligns or orients the model with the topographic map of the patient's skin, or the like, for display during surgery. The model is aligned with the patient's skin in the operating room such that as instruments enter the field of view of the motion-sensing mechanism, the instrument is displayed on the heads up display in surgery in real or near real time.

In still other aspects of the technology, the motion-sensing mechanism is provided with x-ray or magnetic resonance imaging capability to better coordinate the model of the pathology with the patient.

The technology of the present application may be used to identify and track patients, visitors, and/or staff in certain aspects. The motion-sensing mechanisms may make a reference topographic image of the subject's face. In certain embodiments, the reference topographic image may be annotated with information regarding, for example, eye color, hair color, height, weight, etc. Subsequently as the subject passes other motion-sensing mechanisms, a present topographical image is created along with any required annotated information as available. The present topographical image is compared with the database of reference topographical images for a match, which identifies the subject.

In yet other aspects, the technology of the present application may be used for virtual or educational procedures. Moreover, the technology of the present application may be used to remotely control instruments for remote surgery.

In another aspect, the technology may be used to compare the motion of a joint, bones, muscles, tendons, ligaments, or groups thereof to an expected motion of the same. The ability of the actual joint, for example, to move relative to the expect motion may be translated to a range of motion score that can be used to diagnosis treatment options, monitor physical therapy, or the like.

These and other aspects of the technology of the present application will be apparent after consideration of the Detailed Description and Figures herein. It is to be understood, however, that the scope of the application shall be determined by the claims as issued and not by whether given subject matter addresses any or all issues noted in the Background or includes any features or aspects highlighted in this Summary.

### BRIEF DESCRIPTION OF THE DRAWINGS

FIG. 1 is a functional block diagram of an exemplary surgical navigation system;

FIG. 2 is a functional block diagram of an exemplary surgical navigation system;

FIG. 3 is a functional block diagram of an exemplary motion-sensing mechanism of FIG. 2;

FIG. 4 is an exemplary methodology associated with using the technology of the present application;

FIG. 5 is an exemplary methodology associated with using the technology of the present application;

FIG. 6 is an exemplary methodology associated with using the technology of the present application;

FIG. 7 is an exemplary methodology associated with using the technology of the present application;

FIG. 8 is an exemplary methodology associated with using the technology of the present application;

FIG. 9 is a functional block diagram of a system capable of embodying portions of the technology of the present application; and

FIG. 10 is another functional block diagram of a system capable of embodying portions of the technology of the present application.

### DETAILED DESCRIPTION

The technology of the present patent application will now be explained with reference to various figures, tables, and the like. While the technology of the present application is described with respect to neurosurgery and will be described with respect thereto, it will nevertheless be understood that no limitation of the scope of the claimed technology is thereby intended, with such alterations and further modifications in the illustrated device and such further applications of the principles of the claimed technology as illustrated therein being contemplated as would normally occur to one skilled in the art to which the claimed technology relates. Moreover, it will be appreciated that the invention may be used and have particular application in conjunction with other procedures, such as, for example, biopsies, endoscopic procedures, orthopedic surgeries, other medical procedures, and the like in which a tool or device must be accurately positioned in relation to another object whether or not medically oriented.

Moreover, the technology of the present application may be described with respect to certain depth sensing technology, such as, for example, the system currently available from Microsoft, Inc. known as Kinect™ that incorporates technology available from Prime Sense, LTD located in Israel. However, one of ordinary skill in the art on reading the disclosure herein will recognize that other types of sensors may be used as are generally known in the art. Moreover, the technology of the present patent application will be described with reference to certain exemplary embodiments herein. The word “exemplary” is used herein to mean “serving as an example, instance, or illustration.” Any embodiment described herein as “exemplary” is not necessarily to be construed as preferred or advantageous over other embodiments absent a specific indication that such an embodiment is preferred or advantageous over other embodiments. Moreover, in certain instances, only a single “exemplary” embodiment is provided. A single example is not necessarily to be construed as the only embodiment. The detailed description includes specific details for the purpose of providing a thorough understanding of the technology of the present patent application. However, on reading the disclosure, it will be apparent to those skilled in the art that the technology of the present patent application may be practiced with or without these specific details. In some descriptions herein, generally understood structures and devices may be shown in block diagrams to aid in understanding the technology of the present patent application without obscuring the technology herein. In certain instances and examples herein, the term “coupled” or “in communication with” means connected using either a direct link or indirect data link as is generally understood in the art. Moreover, the connections may be wired or wireless, private or public networks, or the like.

As mentioned above, one of the drawbacks associated with current navigational technologies includes registration and tracking of the references, the patient, and the instruments with the image of the patient's anatomy. By way of background, an exemplary conventional tracking system will be explained as it relates to the technology of the present application. Surgical navigation systems including tracking and registration are generally known in the art and will not be explained herein except as necessary for an understanding of the technology of the present application.

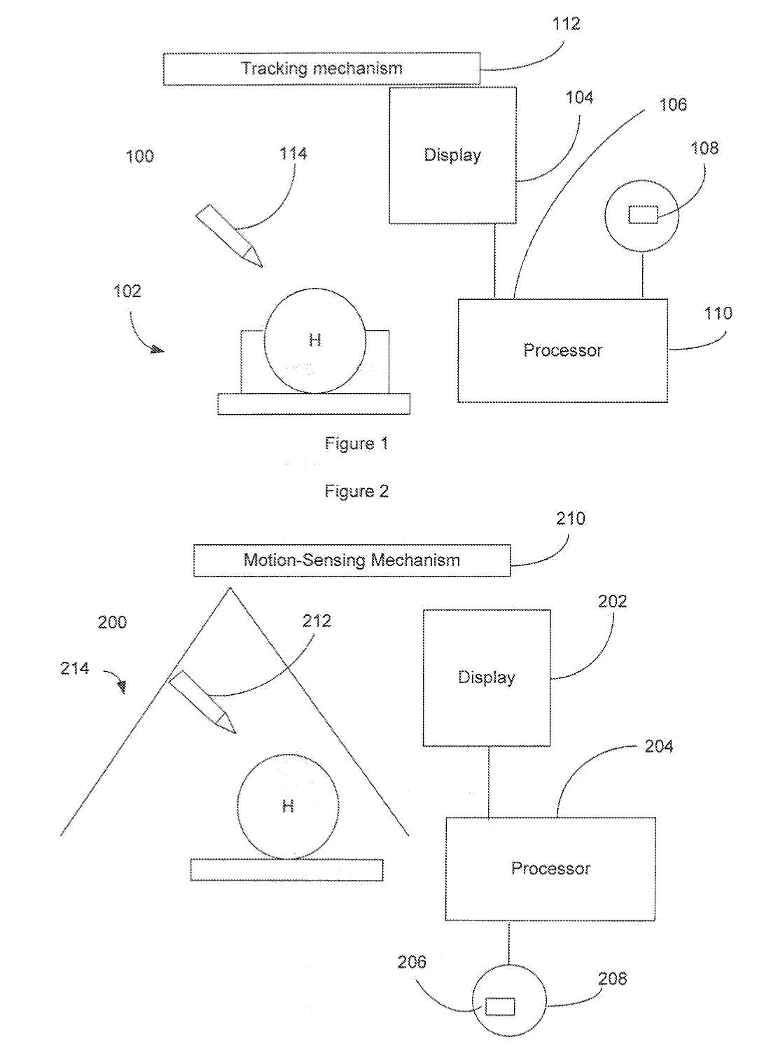

Referring first to FIG. 1, an exemplary surgical navigation system 100 is provided. The surgical navigation system 100 includes, among other things, a reference frame 102 that is placed with specific orientation against a patient's anatomy, such as a head H of the patient. The reference frame 102 may be a head clamp, pins or fasteners implanted to the skull, fiducial markers fixed to the patient's skin, the scalp, or the like. A heads up display 104, such as a high resolution monitor or other device, is coupled to a processor 106. The processor 106 retrieves a model 108 of the patient's anatomy previously developed using conventional navigational techniques from a storage facility 110 and displays the model 108 on the display 104. The model 108 is originally developed using the reference frame 102 such that the orientation of the patient to the instruments may be deduced by the system. Surgical navigation system 100 also includes a tracking mechanism 112 that can locate an instrument 114 in 3-dimensional space. The tracking mechanism 112 may be an optic, sonic, or magnetic system that can identify the location of instrument 114. Conventionally, the instrument 114 is fitted with equipment to allow tracking mechanism 112 to communicate with the instrument 114. Conventionally, a surgeon would register certain instrumentation 114 to be used during the surgical procedure with the system by orienting the instrument 114 with respect to the reference frame 102. The registration process orients or aligns the coordinate system of the patient model 108 to the coordinate system of the instrumentation 114. Once registered, instrument 114 may be tracked with respect to the patient's anatomy and processed by processor 106 such that the position of the instrument 114 is viewable on the display 104 providing the surgeon with a precise image of the instrument within the patient's anatomy.

As can be appreciated, the above system provides numerous issues, some of which have been described above. The registration process is time consuming and can lead to inaccuracies depending on the skill of the surgeon. Only certain instruments are typically fitted such that they can be tracked by tracking mechanism 112. Also, if the patient moves, the orientation to the reference frame may be compromised. This is especially true if the reference frame 102 is secured to the bed frame rather than the patient. Additionally, the added equipment to the instruments and the reference frame often make surgery difficult and awkward.

In accordance with an aspect of the technology of the present application, as will be further explained below, there is provided a system using an object-sensing/depth-sensing device that can be used in surgical procedures to facilitate recognition, registration, localization, mapping, and/or tracking of surgical or other medical instruments, patient anatomy, operating room personnel, patient recognition and/or tracking, remote surgery, training, virtual surgery, and many other applications. Exemplary uses of the technology of the present application further include use in image-guided navigation, image-guided surgery, frameless stereotactic radio surgery, radiation therapy, active vision, computational vision, computerized vision, augmented reality, and the like. The object-sensing mechanism currently contemplated locates points in space based on the distance the point is from the imaging device, e.g., the depth differential of one object to another. The object-sensing mechanism locates objects based on differences in the depth in real-time or near real-time. While the objects located may be stationary, the device processes images in real-time or near real-time and is generically referred to as a motion-sensing mechanism to refer to the fact that the device tracks the movement of objects, instruments, in the field of view.

In one aspect of the technology of the present application, a motion-sensing device may be used to enable a navigation system to identify the relative positions of the targets, such as the patient and the instrument, in 3-dimensional space in order to display their location relative to the patient's radiographic anatomy on a computer monitor. The motion sensing device may use, for example, a depth-sensor to see the targets with or without the use of additional devices, such as, fiducial markers, antenna, or other sensors.

One exemplary device usable with the technology of the present application includes a motion sensing mechanism generally known as KINECT™ available from Microsoft Corporation. This exemplary motion sensing device uses a 3-dimensional camera system developed by PrimeSense Ltd. that interprets information to develop a digitized 3-dimensional model. The motion sensing mechanism includes in one exemplary embodiment an RGB (Red, Green, Blue) camera and a depth sensor. The depth sensor may comprise an infrared laser combined with a monochrome CMOS sensor that captures video data in 3-dimensions. The depth sensor allows tracking multiple tracks in real-time or near real-time. In other exemplary embodiments, the motion-sensing mechanism also may use the RGB camera to enable visual recognition of the targets. In particular, the motion-sensing mechanism would provide a 3-dimensional image of a face, for example, that would be mapped to a previously developed 3-dimensional image of the face. A comparison of the presently recorded image to the data set of pre-recorded images would allow for recognition. In still other exemplary embodiments, the motion-sensing mechanism may be combined with other biometric input devices, such as, microphones for voice/audio recognition, scanners for fingerprint identification or the like.

In one example, the technology of the present application uses the motion-sensing mechanism to enable and facilitate image-guided navigation in surgery or other medical procedures, which can recognize, register, localize, map, and/or track surgical or other medical instruments, patient anatomy, and/or operating room personnel. Optionally, the motion-sensing mechanism may enable and facilitate image-guided navigation in surgery that can track the targets with or without the use of additional devices fixed to the targets, such as those commonly used by prior art and conventional surgical or medical imaging devices, such as, fiducial markers. At least in part because the motion-sensing mechanism does not require instruments to be fitted with tracking sensors or the like, the technology of the present application may track any instrument or object that enters the field being tracked.

In still other examples, the technology of the present application may be used to recognize, register, localize, map, and/or track anatomical features, such as bones, ligaments, tendons, organs, and the like. For example, in one aspect of the technology of the present application, the motion-sensing mechanism may be used for diagnostic purposes by being configured and adapted so as to allow a doctor to assess the extent of ligament damage in an injured joint by manipulating the joint and observing the extent to which the ligament moves as well as noting any ruptures, tears, or other anomalies. In other aspects, the technology could be adapted to be used for diagnostic purposes for a series of joints, such as the human spine, to evaluate motion and various conditions and diseases of the spine. In addition to the diagnostic applications, the motion-sensing mechanism also could be used for therapeutic purposes such as corrective surgery on the joint as well as to monitor and/or measure progress of recovery measures, such as physical therapy with or without surgery. Other therapeutic applications may include using the motion-sensing mechanism to facilitate interventional radiology procedures.

In yet another exemplary use, the technology of the present application may be useful in facilitating the use of navigational technology of computer assisted procedures in medical procedures outside of the operating room. Currently technology is often cost prohibitive for even operating room use. The technology of the present application may facilitate procedures outside the operating room such as beside procedures that may include, for example, lumbar puncture, arterial and central lines, ventriculostomy, and the like. In still further uses, the technology of the present application may be used to establish a reference frame, such as the skull (for ventriculostomy placement) or the clavicle (for subclavian line placements); instead of linking these reference positions to patient specific images, these reference positions could be linked to known anatomical maps; in the exemplary of the ventriculostomy case the motion-tracking mechanism would be used to identify the head and then a standard intracranial image would be mapped to the head. Several options could be picked by the surgeon like a 1 cm subdural, slit ventricle, or the like. This may allow placement without linking the actual patient image to the system. Similar placements may be used for relatively common applications such as line placements, chest tubes, lumbar punctures, or the like where imaging is not required or desired.

In another example, the motion-sensing mechanism facilitates image-guided navigation in surgery so as to track the targets without the mechanical constraints inherent in articulated arms, line-of-sight constraints inherent in conventional infrared light-based tracking systems, and material constraints inherent in the use of magnetic field-based tracking systems.

In still another example, the motion-sensing mechanism may use sound to track and locate targets, which may include voice recognition as identified above. The motion-sensing mechanisms may be configured to use visible or no-visible light or other portions of the electromagnetic spectrum to locate targets, such other portions may include microwaves, radio waves, infrared, etc.

In still another example of operational abilities, the technology of the present application can recognize facial features and/or voice patterns of operating room personnel in order to cue navigation procedures and algorithms.

As explained further below, the technology of the present application may be shown in various functional block diagrams, software modules, non-transitory executable code, or the like. The technology may, however, comprise a single, integrated device or comprise multiple devices operationally connected. Moreover, if multiple devices, each of the multiple devices may be located in a central or remote location. Moreover, the motion-sensing mechanism may be incorporated into a larger surgical navigation system or device.

Available motion-sensing mechanisms include, for example, components currently used in commercially available gaming consoles. For example, components for motion-sensing mechanisms include Wii® as available from Nintendo Co., Ltd; Kinect™, Kinect for Xbox 360™, or Project Natal™ as available from Microsoft Corporation; PlayStation Move™ available from Sony Computer Entertainment Company, and the like. Other commercially produced components or systems that may be adaptable for the technology of the present application include various handheld devices having motion-sensing technology such as gyroscopes, accelerometers, or the like, such as, for example, the iPad™, iPod™, and iPhone™ from Apple, Inc.

With the above in mind, reference is now made to FIG. 2 showing a surgical navigation system 200 consistent with the technology of the present application. The surgical navigation system 200 comprises, similar to the system 100, a heads up display 202 or monitor, such as a high resolution monitor or other device, is coupled to a processor 204. The processor 204 retrieves a model 206 of the patient's anatomy previously developed using CT and MRIs from a storage facility 208 and displays the model 206 on the display 208. The CT and/or MRIs are used in a conventional manner to develop models of the patient's anatomy. The technology of the present application allows the patient's skin (or internal organs, tissue, etc.) to be the reference to orient the model. The surgical navigation system 200 also includes a motion-sensing mechanism 210 that can locate an instrument 212 in 3-dimensional space. The motion-sensing mechanism 210 can identify the location of instrument 212 as will be explained further below. The motion-sensing mechanism 210 also tracks the patient H, which is shown as a head, but could be any portion of the patient's anatomy. The processor would coordinate the model and the patient (which as is explained further below essentially becomes the reference frame 102 because of the ability of the motion-sensing mechanism to track the patient without any additional devices), and the processor aligns the instrument based on the motion-sensing mechanism 210 relative to the patient. Unlike the surgical navigation system 100, the surgeon using the technology associated with surgical navigation system 200 does not need to register the instrument, nor does the instrument need to be fitted with equipment to allow the motion-sensing mechanism to track the instrument. While not shown for convenience, the motion-sensing mechanism 210 may track multiple objects independently allowing the motion-sensing mechanism 210 to track operating room personnel as well as a plurality of instruments 212.

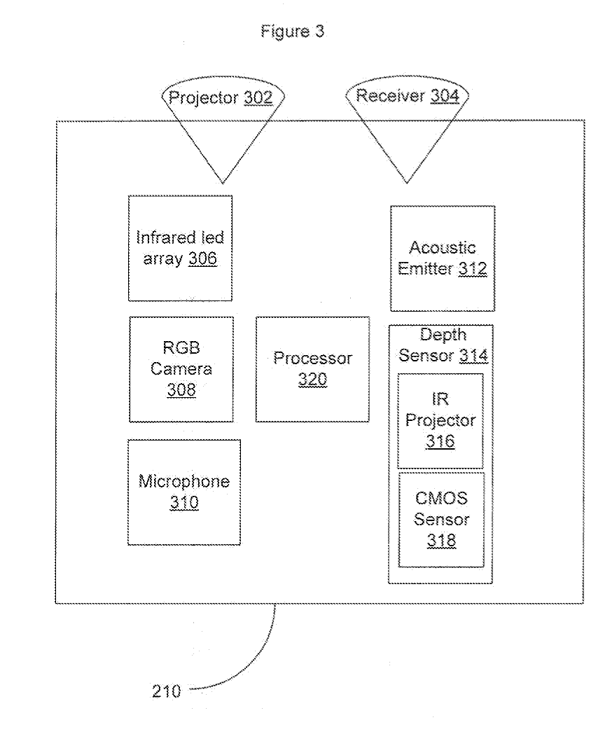

Referring now to FIG. 3, motion-sensing mechanism 210 is shown and described that provides some aspects of the motion-sensing mechanism 210. The motion-sensing mechanism 210 includes a projector 302, a receiver 304, an infrared LED array 306, a RGB camera 308, a multiarray microphone 310, an acoustic emitter 312, a depth sensor 314, which may separately include an infrared projector 316 and monochrome CMOS sensor 318, and a processor 320. As used with reference to FIG. 3, each of the above may include software, hardware, or a combination of software and hardware to facilitate the operation. Moreover, while shown as a combined unit, one or more of the functional block diagram units shown in FIG. 3 may be located in a separate device or remote from the motion-sensing mechanism 210. Additionally, one or more of the functional block diagram units may comprise multiple units or modules and/or one or more of the functional block diagram units may be combined with others of the functional block diagram units.

In certain aspects, the technology of the present application provides a system having components including an RGB camera, a depth sensor, a multi-array microphone, an infrared projector in communication with a monochrome CMOS sensor, a processor, an infrared LED array, an acoustic emitter, a projector, a CPU workstation having associated software. During the system's operation, the system components are operationally connected with one another, either by wires or wirelessly such as by infrared, Wi-Fi™, wireless local area network, Bluetooth™ or other suitable wireless communication technology. When focused on a subject, the system can provide three dimensional views ranging from the surface of the subject's body to its internal regions. The system is further capable of tracking internal and external movements of the subject's (sometimes referred to a patient) body and the movement of other objects within the immediate vicinity of the subject. Additionally, internal and external sounds in the vicinity of the subject can be detected, monitored and associated with the sound's source. Images provided by the system are 3-dimensional, allowing images to penetrate into the subject's body and observe the movement of functioning organs and/or tissues. For example, the efficacy of treating heart arrhythmia with either electric shock or with a pacemaker can be directly observed by viewing the beating heart. Similarly, the functioning of a heart valve also can be observed using the system without physically entering the body cavity. Movement of a knee joint, spine, tendon, ligament, muscle group or the like also can be monitored through the images provided by the system.

Because the system can monitor the movement of articles within the vicinity of the subject, the system can provide a surgeon with 3-dimensional internal structural information of the subject before and during surgery. As a result, a surgical plan can be prepared before surgery begins and implementation of the plan can be monitored during actual surgery. Redevelopment of the model may be required to facilitate visual display on the monitor in the operating room.

The technology of the present application further provides an imaging method that involves (a) providing a subject for imaging, wherein said subject has internal tissues and organs; (b) providing a system having components including a RGB camera, a depth sensor, a multiarray microphone, an infrared projector in communication with a monochrome CMOS sensor, a processor, an infrared LED array, an acoustic emitter, a projector, a CPU workstation having associated software, and a monitor, wherein said components are in communication, one with another; (c) directing the projector onto the subject; and (d) observing on the monitor 3-dimensional images of tissues or organs within the subject in repose or in motion. The method also can be used to observe and monitor the motion of other objects within the vicinity of the subject such as surgical tools and provide 3-dimensional images before, during, and following surgery. The imaging method also can be used for conducting autopsies. Subjects suitable for imaging include members of the animal kingdom, including humans, either living or dead, as well as members of the plant kingdom.

In yet another example, a device is operationally connected to one or more other devices that also may comprise components including an RGB camera, depth sensor, a multiarray microphone, an infrared projector in communication with a monochrome CMOS sensor, a processor, an infrared LED array, an acoustic emitter, a projector, a CPU workstation having associated software. These devices in turn may be operationally connected to and controlled by a master node so as to provide centralized monitoring, feedback, and/or input for multiple procedures occurring either in the same procedure or operating room, in different operating rooms in the same building or campus, or located at multiple locations and facilities.

The technology of the present application will be explained wherein the surgical navigation system 200, for example, is used in conjunction with a CT or MRI system to develop a model of the patient's anatomy or pathology. As explained above, the CT model is developed using cross-sectional slices of the patient and the MRI system stacks images to develop a model that is displayable on the heads up displays described in reference to FIG. 2 above. The motion-sensing mechanism 210 is using, in conjunction with the CT, MRI, or other imaging device, the patient's anatomical features as the reference mechanism. Thus, the model is referenced to, for example, the skin topography of the patient. In certain embodiments and aspects of the technology of the present application, the imaging device may be incorporated into the motion-sensing mechanism 210. This may include mounting the motion-sensing mechanism 210 on a track or rail system such that it may move along or about the patient. Most available motion-sensing mechanisms 210 are stationary and have a field 214 of view in which they are capable of tracking multiple objects, whether stationary or in motion. Orienting or aligning the model of the patient's pathology with the skin topography provides at least one benefit in that the external reference frame may be removed. This reduces surgical time as well as allowing better access for the surgeon to the patient. Additionally, with the patient's anatomy being the reference point for the model, any accidental or intentional movement of the patient will cause the model on the heads up display to orient correctly for the new reference of the patient.

The motion-sensing mechanism 210 has a field 214 of view. As instruments 212, personnel, or other objects enter the field of view 214, the motion-sensing mechanism 210 determines the location of the object with respect to the skin of the patient (or other patient topographic or anatomical reference) and projects the location of the instrument 212 (instrument 212 is used generically to refer to instruments, personnel, or other objects) on the heads up display oriented with respect to the model 206. Some motion-sensing mechanisms 210 may be capable of viewing all 3-dimensions of the instrument 212; however, the motion-sensing mechanism 210 will only register the portion of instrument 212 facing the motion sensing-mechanism 210's projector, for example. Thus, it may be advantageous for the memory 208 to have a database of instruments available to the surgeon. The database may have specification information regarding the various available instruments including, for example, length, width, height, circumference, angles, and the like such that even if only a portion of the instrument is visible, processor 204 or 320 can determine the orientation and hence the location of the entire instrument. In one exemplary embodiment, the processor obtains, for example, a set of dimensions of the visible instrument 212 and compares the same to a database of instrument dimensions stored in memory 208. When the obtained dimensions are matched to the stored dimensions, the processor recognizes the instrument 212 as instrument A having certain known characteristics. Thus, even if only a portion of instrument 212 is visible to the projector, the processor can calculate the location of the non-visible portions of the instruments and display the same on the heads up display with the model with precision. In other aspects of the technology, when an instrument 212 is introduced to the field 214, the surgeon may verbalize (or make some other visual, audio, or combinational gesture) what the instrument 212 is, such as, for example, Stryker Silverglide BioPolar Forceps. The microphone of motion-sensing mechanism 210 would register the verbal acknowledgment of the instrument and equate the instrument 212 introduced to the field 214 as the verbalized instrument.

In still other embodiments, the motion-sensing mechanism 210 includes the depth sensor 314. The depth sensor allows for precise imaging of any particular object to determine the specific external shape of the object or instrument. The entire object can be compared to a database of instrument dimensions to identify the particular instrument. In some embodiments, the instruments are provided with key/unique dimensions that are determinable by the dept sensor 314 in the motion-sensing mechanism 210. The unique dimension is used to identify the particular instrument(s). The system also may register specific instrument information in memory such that when the line of sight to the instrument is blocked in part the processor can use the instrument and vector information to determine the exact location of the instrument or object in three dimensions.

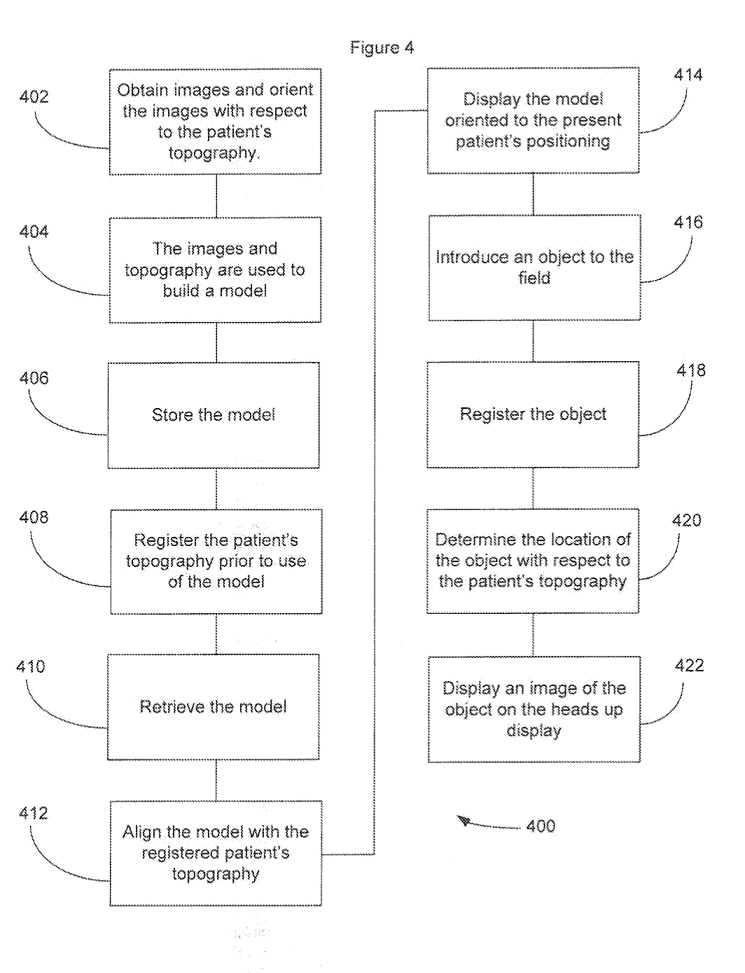

With reference to FIG. 4, an exemplary method 400 of using the technology of the present application is provided. A step 402 includes obtaining the images of the patient's anatomy with reference to the patient's topography as explained above. The images and topography are combined to build a model of the patient's anatomy, step 404. The model is stored for later retrieval during the surgical procedure, step 406. At step 408, motion-sensing mechanism 210 registers the patient's topography prior to the use of the model. At step 410, the model is retrieved from storage. The processor orients the model with the registered patient's topography at step 412. Once oriented, at step 414, the model is displayed referenced to the current positioning of the patient. Next, an object, such as instrument 212, is introduced to the field 214, step 416. Motion-sensing mechanism 210 registers the object, step 418, and determines its 3-dimensional location with respect to the patient's topography, step 420. Once the 3-dimensional location of the object with respect to the patient's topography is identified, the object is displayed on the heads up display, step 422. Optionally, the object is recognized by the processor either automatically by comparing the dimensions of the instrument to a database of instruments or manually by a queue from the surgeon or other operating room personnel. In other aspects of the technology of the present invention, the dimensions of the object are stored after the object is registered by the motion-sensing mechanism 210. During operation, any portions of the object not visualized by the motion-sensing mechanism 210 are calculated by the processor and the actual or calculated position of the object is displayed on the heads up display.

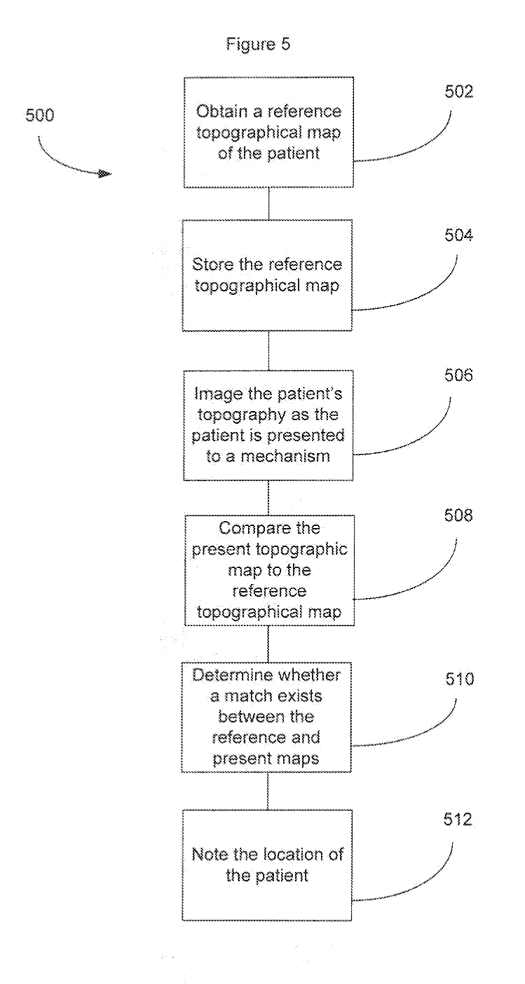

In one aspect of the technology of the present application, as mentioned above, the motion-sensing mechanism may be used to track patients. An exemplary method 500 of using the technology of the present application to track patients is provided in FIG. 5. First, the motion-sensing mechanism may obtain a reference topographical map of the patient's facial features, step 502. The reference topographical map also may include certain features as eye color, hair color, or the like as well as a topographical map. The patient's facial features are stored in a memory, step 504. Next, as a patient is imaged by the same or another motion-sensing mechanism, such as one located in a patient room, a hall way, or a procedural room, the motion-sensing mechanism will make a present topographical map, which may include others of the patient's features as identified above, step 506. The present topographical map is compared to the reference topographical map to determine whether a match is obtained, step 508. If a match is made, the patient's identity is confirmed, step 510, and the location of the patient is noted, step 512. This feature may be useful in many aspects, such as to confirm a patient in an operating room against the patient's registered procedures.

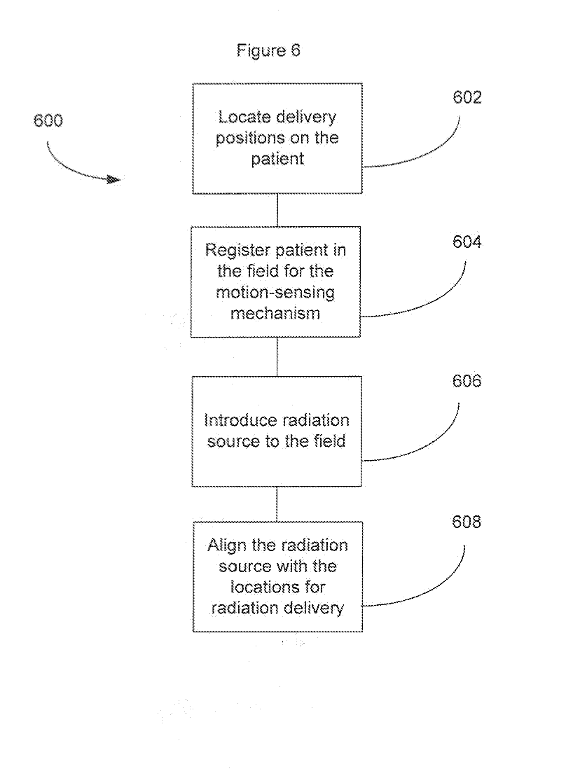

In another aspect of the technology of the present application, the motion-sensing mechanism may be used to align instruments with pre-arranged spots on the patient's anatomy to coordinate delivery of electromagnetic radiation, such as, for example, as may be delivered by stereotactic radio surgical procedures. An exemplary method 600 of using the technology of the present application for delivery of electromagnetic radiation is provided in FIG. 6. First, locations on the patient's skin are located for delivery of a beam of electromagnetic radiation, step 602. Next, the patient is placed in the field 214 and registered by motion-sensing mechanism 210, step 604. The radiation source or emitter is introduced to the field 214 recognized by the motion-sensing mechanism 210, step 606. The motion-sensing mechanism 210 is used to guide each of the radiation sources or emitters to the appropriate alignment with the patient, step 608. The alignment may be automatically provided by robotic actuation.

As can be appreciated, a model of a patient's anatomy may be simulated by the surgical navigation systems described above. The simulated model would allow for virtual surgery and/or training.

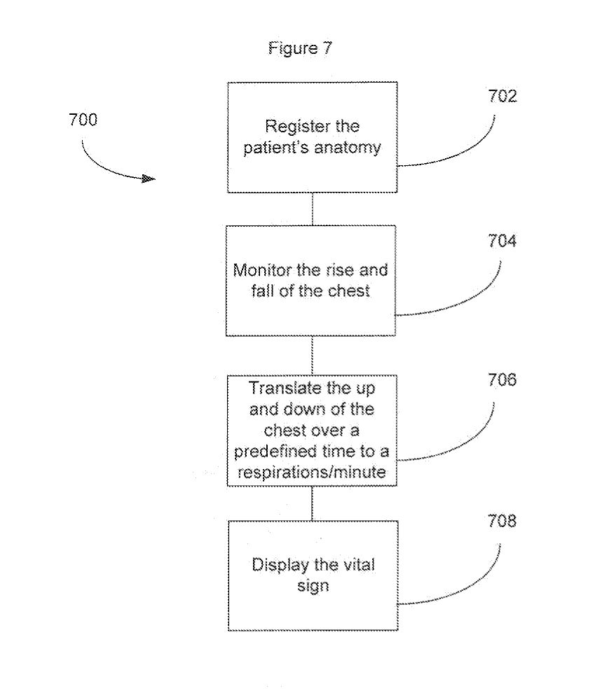

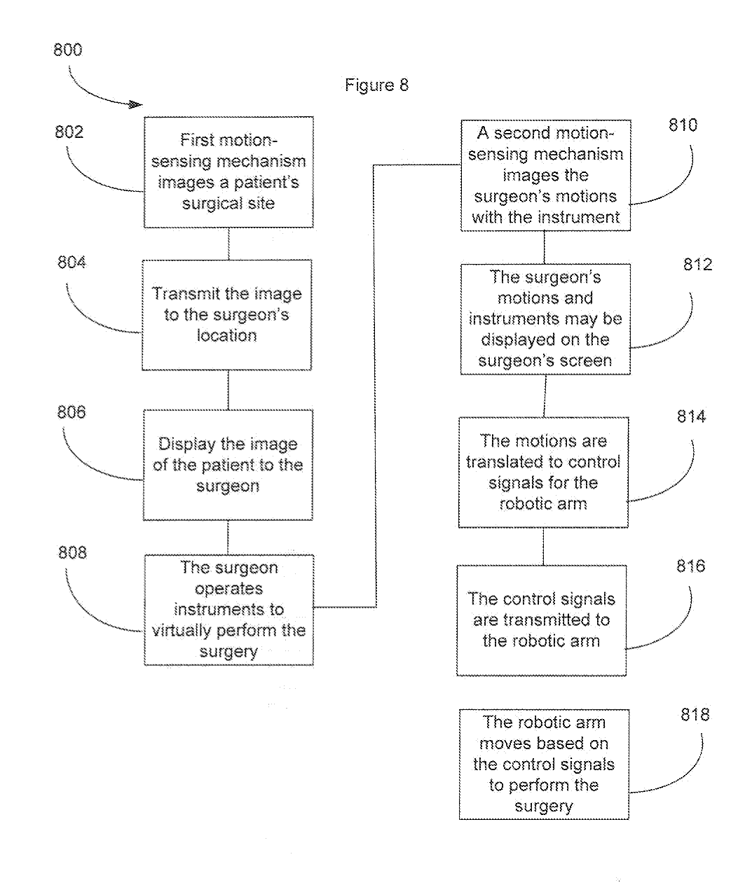

In yet another aspect of the technology of the present application, the motion-sensing mechanism 210 may be used to monitor one or more of a patient's vital signs. An exemplary method 700 of using the technology of the present application for delivery of electromagnetic radiation is provided in FIG. 7. The motion-sensing mechanism 210 registers the patient's anatomy about the chest, step 702. As the chest rises and falls, the motion-sensing mechanism 210 may transmit the motion to the processor, step 704. The processor determines the up and down motion of the chest over a predefined time, step 706, and translates the motion over time into a respirations per minute display on the heads up display, step 708, as the patient's respiration rate. Similarly, the motion-sensing mechanism 210 may be equipped to monitor heart beats per minute, pulse, blood oxygen levels, variable heart rate, skin temperature, or the like.

In still other aspects of the technology of the present application, the motion-sensing mechanism 210 may be used to determine range, strength, function, or other aspects of a patient's anatomy based on comparison of the patient's actual motion compared to an expected or normal range of motion. For example, the spine of a human is expected to have certain range of motion in flexion, extension, medial/lateral, torsion, compression, and tension without or with pain generation and thresholds. The motion-sensing mechanism may be used to monitor the motion of a patient's spine through a series of predefined motions or exercises that mimic a set of motions that are expected by the doctor or health care provider. The actual range of motion through the exercises can be compared to the expected range of motion to determine a result, such as a composite score, that rates the actual spinal motion. For example, a rating of 90-100% may equate to the expected or normal range of motion, 70-80% may equate to below expected, but otherwise adequate motion, where less than 70% may equate to deficient range of motion. The ranges provided and the rating are exemplary. The comparison may be used for other anatomical structures as well such as other bones, tendons, ligaments, joints, muscles, or the like. Other measurements that may be used in a motion based analysis for spinal movement include, for example, flexion velocity, acceleration at a 30° sagittal plane, rotational velocity/acceleration, and the like. The diagnostic may be used to track patient skeletal or spinal movement pre-operatively and/or post-operatively, and compare it to validated normative databases to characterize the movement as consistent or inconsistent with movements expected in certain clinical scenarios. In this way, a clinician may be able to determine if a patient's pain behavior is factitious or appropriately pathologic. This may allow clinicians to avoid treating patients, and/or return treated patients to normal activities, who are malingering.

The range of motion diagnostic may be useful for a number of surgical or non-surgical treatments and therapies. For example, the diagnostic may be used to define the endpoints of treatment. If a patient has a minimally invasive L4/L5 spinal fusion (such as a TLIF), it may be possible to identify recovery when the motion reaches a functional score at or over a predetermined threshold. Moreover, the expected post operative range of motion may be better visualized by patients to appreciate post-operative functioning. The diagnostic could be used to define the progression of treatment. The patient may go to conservative care, but a serial functional test shows there is no improvement. Instead of extending the conservative care for months, once the functional motion diagnostics shows no progression on motion/pain, the patient can make the decision for more aggressive treatment sooner. Also, even with progression, the motion diagnostic could be used to determine when recovery is sufficient to terminate physical therapy or the like.

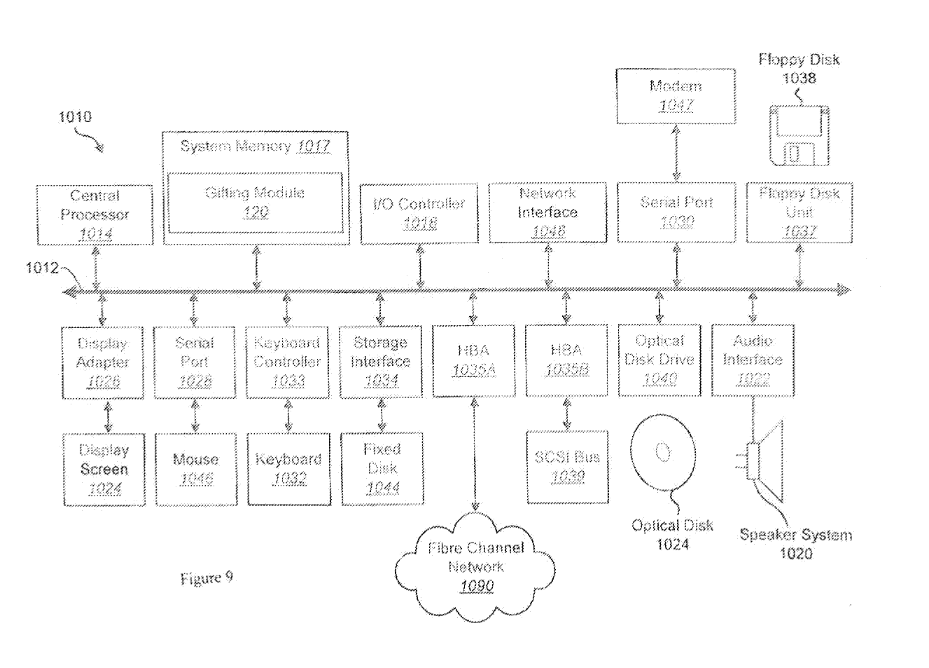

In yet another aspect of the technology of the present application, the surgical navigation system 200 or the like may be used in remote or robotic surgery. An exemplary method 800 associated with using the technology for remote or robotic surgery is provided in FIG. 8. Remote surgery may or may not use the heads up display, but for convenience will be explained herein with reference to the surgical procedure allowing the surgeon to remotely visualize the patient. Initially in this exemplary methodology, a first motion-sensing mechanism is used to image a patient including the surgical site, step 802. The image of the patient is transmitted from the motion-sensing mechanism to a surgeon screen that is established remotely, step 804. The image is displayed to the surgeon on the screen, step 806. The screen may be a conventional monitor, a holographic image, or a visor screen. The surgeon would operate instruments based on the visual image for the particular surgery, step 808. A second motion-sensing mechanism would image the surgeon's movements including the selection of particular instruments, step 810. The second motion-sensing mechanism may display the surgeon's movements with the instruments on the surgeon's visual image, step 812. A processor would translate the surgeon's movements into control signals for a robotic arm located at the surgical site, step 814. The processor would transmit the control signals to the robotic arm located proximate the patient, step 816. Finally, the robotic arm would perform the surgical movements using the same instrument the remote surgeon has selected to perform the surgery, step 818.

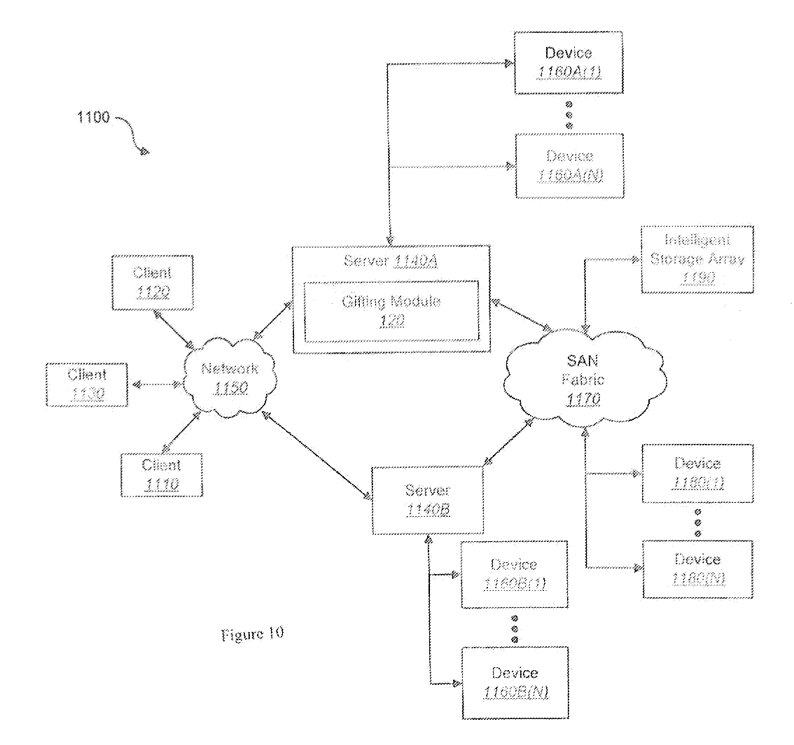

FIG. 9 depicts a block diagram of a computer system 1010 suitable for implementing the present systems and methods. Computer system 1010 includes a bus 1012 which interconnects major subsystems of computer system 1010, such as a central processor 1014, a system memory 1017 (typically RAM, but which may also include ROM, flash RAM, or the like), an input/output controller 1018, an external audio device, such as a speaker system 1020 via an audio output interface 1022, an external device, such as a display screen 1024 via display adapter 1026, serial ports 1028 and 1030, a keyboard 1032 (interfaced with a keyboard controller 1033), multiple USB devices 1092 (interfaced with a USB controller 1090), a storage interface 1034, a floppy disk drive 1037 operative to receive a floppy disk 1038, a host bus adapter (HBA) interface card 1035A operative to connect with a Fibre Channel network 1090, a host bus adapter (HBA) interface card 10356 operative to connect to a SCSI bus 1039, and an optical disk drive 1040 operative to receive an optical disk 1042. Also included are a mouse 1046 (or other point-and-click device, coupled to bus 1012 via serial port 1028), a modem 1047 (coupled to bus 1012 via serial port 1030), and a network interface 1048 (coupled directly to bus 1012).

Bus 1012 allows data communication between central processor 1014 and system memory 1017, which may include read-only memory (ROM) or flash memory (neither shown), and random access memory (RAM) (not shown), as previously noted. The RAM is generally the main memory into which the operating system and application programs are loaded. The ROM or flash memory can contain, among other codes, the Basic Input-Output system (BIOS) which controls basic hardware operation such as the interaction with peripheral components or devices. For example, the gifting module 104 to implement the present systems and methods may be stored within the system memory 1017. Applications resident with computer system 1010 are generally stored on and accessed via a computer readable medium, such as a hard disk drive (e.g., fixed disk 1044), an optical drive (e.g., optical drive 1040), a floppy disk unit 1037, or other storage medium. Additionally, applications can be in the form of electronic signals modulated in accordance with the application and data communication technology when accessed via network modem 1047 or interface 1048.

Storage interface 1034, as with the other storage interfaces of computer system 1010, can connect to a standard computer readable medium for storage and/or retrieval of information, such as a fixed disk drive 1044. Fixed disk drive 1044 may be a part of computer system 1010 or may be separate and accessed through other interface systems. Modem 1047 may provide a direct connection to a remote server via a telephone link or to the Internet via an Internet service provider (ISP). Network interface 1048 may provide a direct connection to a remote server via a direct network link to the Internet via a POP (point of presence). Network interface 1048 may provide such connection using wireless techniques, including digital cellular telephone connection, Cellular Digital Packet Data (CDPD) connection, digital satellite data connection or the like.

Many other devices or subsystems (not shown) may be connected in a similar manner (e.g., document scanners, digital cameras and so on). Conversely, all of the devices shown in FIG. 9 need not be present to practice the present systems and methods. The devices and subsystems can be interconnected in different ways from that shown in FIG. 9. The operation of a computer system, such as that shown in FIG. 9, is readily known in the art and is not discussed in detail in this application. Code to implement the present disclosure can be stored in computer-readable medium such as one or more of system memory 1017, fixed disk 1044, optical disk 1042, or floppy disk 1038. The operating system provided on computer system 1010 may be MS-DOS®, MS-WINDOWS®, OS/2®, UNIX®, Linux®, or another known operating system.

FIG. 10 is a block diagram depicting a network architecture 1100 in which client systems 1110, 1120 and 1130, as well as storage servers 1140A and 1140B (any of which can be implemented using computer system 1110), are coupled to a network 1150. In one embodiment, the gifting module 104 may be located within a server 1140A, 1140B to implement the present systems and methods. The storage server 1140A is further depicted as having storage devices 1160A(1)-(N) directly attached, and storage server 1140B is depicted with storage devices 1160B(1)-(N) directly attached. SAN fabric 1170 supports access to storage devices 1180(1)-(N) by storage servers 1140A and 1140B, and so by client systems 1110, 1120 and 1130 via network 1150. Intelligent storage array 1190 is also shown as an example of a specific storage device accessible via SAN fabric 1170.

With reference to computer system 1010, modem 1047, network interface 1048 or some other method can be used to provide connectivity from each of client computer systems 1110, 1120, and 1130 to network 1150. Client systems 1110, 1120, and 1130 are able to access information on storage server 1140A or 11408 using, for example, a web browser or other client software (not shown). Such a client allows client systems 1110, 1120, and 1130 to access data hosted by storage server 1140A or 1140B or one of storage devices 1160A(1)-(N), 1160B(1)-(N), 1180(1)-(N) or intelligent storage array 1190. FIG. 10 depicts the use of a network, such as the Internet, for exchanging data, but the present systems and methods are not limited to the Internet or any particular network-based environment.

While the foregoing disclosure sets forth various embodiments using specific block diagrams, flowcharts, and examples, each block diagram component, flowchart step, operation, and/or component described and/or illustrated herein may be implemented, individually and/or collectively, using a wide range of hardware, software, or firmware (or any combination thereof) configurations. In addition, any disclosure of components contained within other components should be considered exemplary in nature since many other architectures can be implemented to achieve the same functionality.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various exemplary methods described and/or illustrated herein may also omit one or more of the steps described or illustrated herein or include additional steps in addition to those disclosed.

Those of skill in the art would understand that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Those of skill would further appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The various illustrative logical blocks, modules, and circuits described in connection with the embodiments disclosed herein may be implemented or performed with a general purpose processor, a Digital Signal Processor (DSP), an Application Specific Integrated Circuit (ASIC), a Field Programmable Gate Array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by a processor, or in a combination of the two. A software module may reside in Random Access Memory (RAM), flash memory, Read Only Memory (ROM), Electrically Programmable ROM (EPROM), Electrically Erasable Programmable ROM (EEPROM), registers, hard disk, a removable disk, a CD-ROM, or any other form of storage medium known in the art. An exemplary storage medium is coupled to the processor such that the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

We claim:

1. An apparatus comprising:
a processor;
a memory coupled to the processor to store a model of anatomical pathology of a patient;
a motion-sensing mechanism having a depth sensor coupled to the processor, the motion-sensing mechanism adapted to register the location of a patient's topography in a field and adapted to track movement of an object in the field relative to the patient's topography using the depth sensor to determine relative distances and translate the movement into position information; and
a display coupled to the processor,
wherein the processor fetches the model from the memory and displays the model relative to the patient's topography and the processor retrieves the position information and displays the object relative to the patient's topography and the model.

2. The apparatus of claim 1 wherein the motion-sensing mechanism comprises a projector and a receiver that cooperate to track a plurality of objects in the field.

3. The apparatus of claim 2 wherein the projector is an x-ray emitter.

4. The apparatus of claim 2 wherein the projector is an electromagnet.

5. The apparatus of claim 1 further comprising a microphone.

6. The apparatus of claim 1 further comprising:
a projector and receiver to image the anatomical pathology of the patient.

7. The apparatus of claim 6 wherein the projector and receiver are selected from a group of projectors and receivers consisting of: x-rays, electromagnetic, infrared, or sonic.

8. The apparatus of claim 2 wherein the projector is an infrared light emitter.

9. The apparatus of claim 2 wherein the receiver is a depth-sensing receiver.

10. A method useful for computer assisted surgery, the method performed on at least one processor comprising the steps of:
creating a model of a patient's anatomy from images of the patient's anatomy obtained prior to a surgical procedure;
registering a patient's topography in an operating room using a motion-sensing mechanism;
aligning the patient's topography and the model;
displaying the model aligned with the patient's topography on a display in an operating room;
tracking an object in a surgical field using a motion-sensing mechanism;
identifying a location of the object relative to the patient's topography; and
imaging the object on the display along with the model to facilitate the surgical procedure.

11. The method of claim 10 wherein the step of creating the model of the patient's anatomy comprises using magnetic resonance images.

12. The method of claim 10 wherein the step of creating the model of the patient's anatomy comprises using x-ray cross-sections of the patient.
